(19) United States
(12) Patent Application Publication
(10) Pub. No.: US 2024/0350704 A1
(43) Pub. Date: Oct. 24, 2024

(54) IMPLANT

(71) Applicant: THE UNIVERSITY OF BIRMINGHAM, Birmingham (GB)

(72) Inventor: Gowsihan POOLOGASUNDARAMPILLAI, Birmingham, West Midlands (GB)

(21) Appl. No.: 18/701,733

(22) PCT Filed: Oct. 20, 2022
(86) PCT No.: PCT/GB2022/052675

§ 371 Date: Apr. 16, 2024

(30) Foreign Application Priority Data
Oct. 20, 2021 (GB) 2115042.0

(51) Int. Cl. A61L 27/10; A61L 27/38

(52) U.S. Cl. CPC A61L 27/10; A61L 27/3847; A61L 2300/104; A61L 2300/112; A61L 2430/02

(57) ABSTRACT

A method of forming an implant for the repair of defects in bone. the method comprises the steps of: electrospinning bioactive glass fibres: compressing the electrospun bioactive glass fibres to form a compressed body: heating the compressed body to bond the fibres to form a shaped body: heat treating the shaped body to form a heat-treated shaped body. There is also disclosed an implant for use in repairing critical or sub critical bone defects.

This invention relates generally to an implant. More specifically, although not exclusively, this invention relates to an implant comprising bioactive glass.

In surgical reconstruction procedures it may be necessary to insert graft materials into a defect site to facilitate healing. Grafts from a different site of the patient (autografts) are commonly used as they allow for fast healing and, for obvious reasons, have low rejection rates. However, such procedures are limited by the available bone-graft volume and are associated with donor site morbidity. Allografts, xenografts or synthetic bone grafts are alternatives but these lack the osteogenic and angiogenic properties of autografts.

Cancellous bone allo-or xenograft granules possess a micro-porous structure and high surface area but, once packed into a defect, this property is lost. Usually capillaries will penetrate no further than a few millimetres from the surface. Further, the packed granules often lack the required mechanical stability to maintain graft shape and volume in unconfined defects, thereby suffering from post-operative graft mobility which is likely to delay tissue healing.

In order for a wound to heal there must be tissue regeneration and for this to occur successfully a scaffold is often introduced to encourage natural healing. An ideal scaffold is a three dimensional (3-D) scaffold that mimics the extracellular matrix (ECM) of the tissue.

Clinically available products are either particulate, which do not mechanically support the defect environment, or 3-D porous structures which are not bioactive and hence result in poor bone formation.

Bioactive glasses are a unique class of synthetic biomaterials. Bioactive glasses can bond to tissue and stimulate new tissue formation via the release of ionic species such as silica and Ca²⁺as well as other ions. It is known that bioactive glasses if doped with Ag²⁺, Cu²⁺and Zn²⁺can give rise to an antibacterial effect (for example see J. Biomed. Mat. Res. (2000), 51(3) 484-490). Further, doping bioactive glasses with strontium has been shown to promote bone formation (e.g. ACS Appl. Mater. Interfaces (2018), 10, 27, 23311-23320). Copper and cobalt are also known to promote angiogenesis (for example see J. Mater. Chem. B, (2013), 1, 5659-5674).

Bioactive glasses have tremendous potential for clinical use. However, due to the difficulty in producing 3-D porous structures, in particular a 3-D scaffold that mimics the ECM, they have not found significant clinical applications. Several groups have developed methods to produce bioactive glasses with a 3-D porous architecture to act as scaffolds for tissue growth. However, once implanted these 3-D scaffolds have limited ability for perfusion and are brittle.

The most commercially successful bioglass is 45S5 (hereinafter BioGlass—marketed by PerioGlas, Biogran®, Unigraft® and GlassBone), a first-generation glass produced by melt-quenching oxide components from high temperature (>1400° C.). However, this limits the physical form of 4S5S to either powder, granular or large fibres (>20 μm).

ETS Mirragen® is a fibrous bioactive glass. The fibres are formed by directing a jet of gas on to a stream of glass melt. This draws the fibres while also cooling and solidifying to an amorphous glass. Although this can be used to produce BioGlass fibres with smaller diameter (<20 μm) the technique is very chaotic leading to an inhomogeneous product. Further, biomolecules which could transform wound treatments, or organics which could offer toughening and flexibility, cannot be loaded since the fibres are formed from a melt at 1400° C.

Geistlich Bio-Oss® is a hard, granular material that is osteoconductive however it does not degrade as new bone is formed, it also does not provide a mechanically stable environment. Further, because it is of animal origin there are batch-to-batch variations which may result in immunogenicity.

Maxresorb® from Botiss Biomaterials GmbH is a granular, fully synthetic, bone substitute material, the granules having a hydrophilic surface and being composed of 60% hydroxyapatite (HA) and 40% beta tri-calcium phosphate (β-TCP), the HA being resorbable at a slower rate than the β-TCP.

Further, procedures requiring lateral and vertical bone augmentation cannot be regenerated by particulate materials alone. As such, titanium meshes are often employed to preserve the shape of the particulate implant until new bone has formed.

In particular successful bone regeneration in defects of the jaw still present a significantly unmet clinical need. For example, in challenging (and yet common) clinical cases where the alveolar ridge of the mandible/maxilla requires lateral and vertical augmentation a self-supporting 3D interconnected porous structure will have considerable advantages.

It is therefore a first non-exclusive object of the invention to provide synthetic graft material or implant that provides a mechanically stable environment.

It may be a further objective to provide a synthetic bone graft material which has one or more of a perfusable interconnected porous structure, which degrades allowing new bone to grow, which is bioactive (in order to allow it to bond with existing bone and chemoattract cells for osteogenesis and angiogenesis), which is self-supporting, flexible and/or conformable (to allow manipulation into wounds that are irregular in shape and/or size) and/or is ethically, culturally and/or religiously acceptable to patients.

A further object of the invention is to provide a synthetic bone graft usable in surgery, in particular maxillofacial and oral surgery, particularly surgical reconstructions (e.g. maxillofacial and oral surgery reconstructions) where there is a sub critical or critical defect (a critical defect being defined as one that will not heal spontaneously during a patient's lifetime).

Accordingly, a first aspect of the invention provides a method of forming an implant for the repair of defects in bone, the method comprising:
(a) electrospinning bioactive glass fibres;
(b) compressing the electrospun bioactive glass fibres to form a compressed body;
(c) heating the compressed body to bond the fibres to form a shaped body;
(d) heat treating the shaped body to form a heat-treated shaped body
(e) optionally, shaping the heat-treated shaped body into an implant.

Step (b) may comprise compressing the electrospun bioactive glass fibres to a density of 0.02 to 0.3 mg/mm³.

Step (b) may comprise compressing the electrospun bioactive glass fibres to a density of 0.03 to 0.30 mg/mm³, for example from one of 0.03, 0.04, 0.05, 0.06, 0.07, 0.08, 0.09, 0.10, 0.11, 0.12, 0.13, 0.14, 0.15, 0.16, 0.18 mg/mm³ to one of 0.30, 0.29, 0.28, 0.27, 0.26, 0.25, 0.24, 0.23, 0.22, 0.21, 0.20, 0.19, 0.18, 0.17, 0.16, 0.15, 0.14, 0.13, 0.12, 0.11, 0.10, 0.09, 0.08, 0.07, 0.06, 0.05, 0.04 mg/mm³. Step (b) may comprise compressing the electrospun bioactive glass fibres to a density of 0.05 to 0.29 mg/mm³.

Step (b) may comprise compressing the electrospun bioactive glass fibres in a die or mould.

It has been found that cell ingrowth and migration within the so formed implant or heat-treated shaped body may be optimised when the electrospun bioactive glass fibres are compressed to a density of 0.05 or 0.10 to 0.20 mg/mm³, for example from 0.05 to 0.20 mg/mm³, say from 0.05 or 0.12 to 0.18 mg/mm³. For example, we have found that densities of 0.05 to 0.10 mg/mm³ may be particularly beneficial for bone ingrowth for critical and sub-critical bone defects in vivo.

Step (c) may comprise heating to a temperature of less than 100° C.

Step (c) may comprise heating to a temperature of less than or equal to 95, 90, 85, 80, 75, 70° C. Step (c) may comprise heating to a temperature of greater than or equal to 30, 35, 40, 45, 50, 55, 60, 65, 70, 75, 80, 85, 90, 95° C. Step (c) may comprise heating to a temperature of from one or more of 30, 35, 40, 45, 50, 55, 60, 65° C. to one or more of 95, 90, 85, 80, 75, 70° C. For example, Step (c) may comprise heating to a temperature of from 30 to 95° C., say from 40 to 80° C., for example from 50 to 75° C. In an example, step (c) may comprise heating to a temperature of 60 to 70° C., say 65° C.

Step (c) may comprise heating the electrospun bioactive glass fibres under compression.

Step (c) may comprise heating the electrospun bioactive glass fibres for in excess of 30 minutes, for example 1, 1.5, 2. 2.5, 3, 3.5 or 4 hours.

Step (c) may comprise heating the electrospun bioactive glass fibres in a or the die or mould.

Advantageously it has been found that heating can promote, encourage and/or cause the formation of connected or interlocked fibres so that the so-formed shaped body retains its shape when not under compression and has a wicking effect. Thus, the compressed body is shape-stable and handleable.

We have also found that conducting step (c) before step (d) is beneficial because it allows the fibres to form desired (e.g. complex) shapes whereas heat treating then moulding or forming (i.e. step (d) before step (c)) inevitably leads to fibre breakage and a less stable structure because of, at least in part, the increased modulus of the fibres.

Step (d) may comprise heat treating the shaped body at a temperature of above 250° C.

For example, Step (d) may comprise heat treating the shaped body at a temperature of above 260, 265, 270, 275, 280, 285, 290, 295, 300° C.

Step (d) may comprise heat treating the shaped body at first temperature for a first period and at a second temperature for a second period. The first temperature may be higher or lower than the second temperature. The first period may be shorter, longer or the same as the second period.

In an example, Step (d) may comprise heat treating the shaped body at a temperature in excess of 250° C. for a first period and at in excess of 400° C. for a second period.

Step (d) may comprise heat treating the shaped body for in excess of 30 minutes, for example in excess of 40, 50, 60, 70, 80, 90, 100, 120, 140, 160, 180, 200, 220, 240, 260, 280, 300, 320, 340, 360, 380, 400 minutes. Step (d) may comprise heat treating the shaped body for less than 120, 140, 160, 180, 200, 220, 240, 260, 280, 300, 320, 340, 360, 380, 400, 420, 440, 460, 480, 500, 520, 540, 560, 580, 600, 620, 640, 660, 680, 700 minutes.

The first period may be from 10 minutes to 120 minutes. The second period may be from 120 to 400 minutes.

In an example, Step (d) may comprise heat treating at 300° C. for 30 minutes and then heat treating at 650° C. for 300 minutes.

The temperature may be increased between the first period and the second period at a steady ramp rate, for example a ramp rate of from 0.5 to 20° C./min. It may be preferred that the ramp rate is less than 20° C./min, for example between 0.5 to one of 19, 18, 17, 16, 15, 14, 13, 12, 11, 10, 9, 8, 7, 6 or 5° C./min.

Step (d) may comprise calcining the fibres of the shaped body.

Step (d) may comprise heat treating the shaped body in a compressed or uncompressed state.

Step (d) is performed below a temperature sufficient to cause viscous flow in the fibres (say lower than 700, 690, 680, 670, 660° C.). Heat treating at temperatures below the viscous flow limit allows the organic materials to be removed and ensures that (or at least limits the opportunity for) the individual fibres do not melt and flow together to form a glassy or amorphous matrix. We believe that this is beneficial because it retains the shape of the implant, the individual nature of the fibres and the porosity of the fibres. This, we believe, contributes to the efficacy of the implant in vivo, that is to the resorbability of the implant (heat-treated shaped body), the rate of bone ingrowth, the nature of the ingrowing bone and/or the degree of vascularization in the newly formed bone. In contrast, materials which have been heated above the viscous flow temperature tend to have a glassy, shiny or reflective surface. As a consequence of that heating they tend to be less porous and, as such, a less likely to allow for ingrowth and/or to be rabidly resorbed.

Step (d) need not be conducted under compression. In embodiments step (c) is conducted under compression but step (d) is not. As such, at least some, most, or all compressive forces may be removed before step (d) is undertaken. For example, a closure for a mould may be removed between steps (c) and (d), or the shaped body may be removed from a mould after step (c) to remove all retention. It will be appreciated that upon heat-treating the shaped body it will shrink due to removal of the organic materials. As such, retention within a mould is not required for the heat-treating step (d).

It has been found that heat treating allows for the formation of a porous structure within the fibres. The pores may have diameters of less than 200 nm, for example less than or equal to 190, 180, 170, 160, 150, 140, 130, 120, 110, 100 nm. The pores may have diameters in excess of 2 nm, for example greater than or equal to 2, 3, 4, 5, 6, 7, 8, 9, 10, 15, 20, 25, 30, 35, 40, 45, 50 nm. In an example, the pores have diameters of form 1 to 80 nm, for example from 2 to 70 nm. In one example the pores have diameters for from 5 to 55 nm.

Step (e) may comprise shaping, e.g. cutting, the heat-treated shaped body to form a required shape and/or sized implant. For example, the heat-treated shaped body may be in the form of a sheet and the implant may be cut from the sheet. Additionally plural layers of the heat-treated shaped body may be combined (e.g. stacked) and provided as an implant. Alternatively, the shaped body of step (c) may be near net shape and the heat treatment of step (d) may cause the shaped body to adopt, as the heat-treated shaped body, the desired net shape.

The method may further comprise sterilising the heat-treated shaped body, for example by exposing the heat-treated shaped body to a temperature of in excess of 150° C. for in excess of 100 minutes. The heat-treated shaped body and/or the sterilised heat-treated shaped body may be packed or packaged under aseptic or sterile conditions.

The method may comprise a preliminary step of forming a sol-gel.

The method may also comprise contacting and/or impregnating the heat-treated shaped body (and/or the sterilised heat-treated shaped body) with cells, for example stem cells. In a particular example the heat-treated shaped body may be contacted and/or impregnated with mesenchymal stem cells (MSCs).

The method may also comprise contacting and/or impregnating the heat-treated shaped body (and/or the sterilised heat-treated shaped body) with chemical agents, for example one or more of antibiotics, medicaments, proteins, hormones (for example growth factors) enzymes, co-factors blood and or components thereof, for example plasma, marrow aspirate, metal ions, salts, nanoparticles and so on.

A further aspect of the invention provides an implant for the repair of defects in bone, the implant comprising a compressed mass of cross linked or bonded, non-woven and preferably non-layered nanofibers, the nanofibers comprising heat treated bioactive glass fibres.

The implant or the heat-treated shaped body may have a density of 0.02 to 0.3 mg/mm³.

In particular, the implant and/or the bioactive glass fibres may have a density of 0.03 to 0.30 mg/mm³, for example from one of 0.03, 0.04, 0.05, 0.06, 0.07, 0.08, 0.09, 0.10, 0.11, 0.12, 0.13, 0.14, 0.15, 0.16, 0.18 mg/mm³ to one of 0.30, 0.29, 0.28, 0.27, 0.26, 0.25, 0.24, 0.23, 0.22, 0.21, 0.20, 0.19, 0.18, 0.17, 0.16, 0.15, 0.14, 0.13, 0.12, 0.11, 0.10, 0.09 mg/mm³. In an example the bioactive glass fibres have a density of 0.05 to 0.29 mg/mm³. In examples where the implant has absorbed other species, for example, blood or blood products (plasma or enriched plasma) the density will be higher depending on the amount of absorbed material.

A yet further aspect of the invention provides an implant for the repair of defects in bone, the implant comprising non-woven bonded bioactive glass fibres having a density of 0.05 to 0.29 mg/mm³.

For example, the non-woven bonded bioactive glass fibres have a density of from one of 0.05, 0.06, 0.07, 0.08, 0.09, 0.10, 0.11, 0.12, 0.13, 0.14, 0.15, 0.16, 0.18 mg/mm³ to one of 0.29, 0.28, 0.27, 0.26, 0.25, 0.24, 0.23, 0.22, 0.21, 0.20, 0.19, 0.18, 0.17, 0.16, 0.15, 0.14, 0.13, 0.12, 0.11, 0.10, 0.09 mg/mm³.

The bioactive glass fibres may have a mean diameter (d50) of from 500 to 1500 nm, for example from 600 to 1200 nm, for example less than 1000 nm.

The bioactive glass fibres may be calcined. The bioactive glass fibres may be porous. The pores may have diameters of less than 200 nm, for example less than or equal to 190, 180, 170, 160, 150, 140, 130, 120, 110, 100 nm. The pores may have diameters in excess of 2 nm, for example greater than or equal to 2, 3, 4, 5, 6, 7, 8, 9, 10, 15, 20, 25, 30, 35, 40, 45, 50 nm. In an example, the pores have diameters of form 1 to 80 nm, for example from 2 to 70 nm. In one example the pores have diameters for from 6 to 55 nm.

The implant may have a thickness of from 0.1 to 20 mm, for example from 0.1 mm to one of 19, 18, 17, 16, 15 mm. The implant may have a thickness of from 1 to 20 mm, for example from 1, 2, 3, 4, or 5, to any one of 20, 19, 18, 17, 16, 15 mm. Other sizes are of course possible, depending on the size of the defect. However, for ease of use, manipulation and conformation, a maximum thickness of 20 mm may be preferred in many if not most surgical settings.

The bioactive glass fibres may comprise SiO₂ and CaO. The bioactive glass fibres may comprise a greater weight present of SiO₂ than CaO. In an example the weight proportion of SiO₂ may be from 40 to 85 w/w %. The weight percentage of CaO may be from 15 to 35 w/w %. In an embodiment the bioactive glass fibres may comprise oxides of one or more of sodium, potassium, magnesium, phosphorous. In an embodiment the bioactive glass fibres may be composed of 45 wt % SiO₂, 24.5 wt % CaO, 24.5 wt % Na₂O, and 6.0 wt % P₂O₅ (i.e. 45S5 bioactive glass). In another embodiment the bioactive glass fibres may be composed of ca 70 w/w % SiO₂ and ca 30 w/w % CaO (e.g. 70 mol % SiO₂ and ca 30 mol % CaO).

Additionally or alternatively, the bioactive glass fibres may comprise or may be doped with one or more of copper, silver, zinc and/or cobalt (for example using one or more of the papers set out above).

The implant may comprise cells, for example stem cells. In a particular example the implant may be impregnated with cells, for example with stem cells, an example being mesenchymal stem cells (MSCs).

The implant may comprise chemical agents, for example one or more of antibiotics, medicaments, proteins, hormones (for example growth factors) enzymes, co-factors blood and or components thereof, for example plasma, marrow aspirate, metal ions, salts, nanoparticles and so on. The implant may be impregnated with chemical agents, for example one or more of antibiotics, medicaments, proteins, hormones (for example growth factors) enzymes, co-factors and so on.

The implant may be packaged within aseptic or sterile packaging.

Advantageously, as the biowool is synthetic, it does not have any animal origin, meaning it is ethically, culturally and/or religiously acceptable to patients.

A further aspect of the invention provides repairing a bone defect, the method comprising locating an implant in the defect, the implant comprising non-woven, bonded bioactive glass fibres having a density of 0.05 to 0.29 mg/mm³.

The bone defect may be a critical or sub critical bone defect, in particular a maxillofacial or oral bone defect. The bone defect may be within a non-load-bearing bone.

Additionally or alternatively, the implant or heat-treated shaped body may be located around a prosthesis. For example, where, say, a hip or knee joint is being replaced a hip or knee prosthesis has to be sited within existing bone. This typically entails removing some of the existing bone. The implant or heat-treated shaped body may be located around at least a portion of the prosthesis to better encourage bone growth from the existing or remaining bone.

The implants are typically free of a consolidating matrix, such as a polymer matrix, which would at least partially fill the interstitial spaces between fibres and/or provide at least some structural rigidity (we call such implants ‘matrix free’). We believe that this makes conformability and control of ingrowth and/or resorption of the implants of the invention a matter of controlling one or more of fibre chemistry, porosity, fibre shape and implant size and density. The implants are self-supporting, without a consolidating matrix, meaning that such implants are handleable (and formable) in a surgical environment, in contrast to powdered or granular materials.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

In order that the invention may be more fully understood, it will now be described, by way of example only, with reference to the accompanying drawings in which:

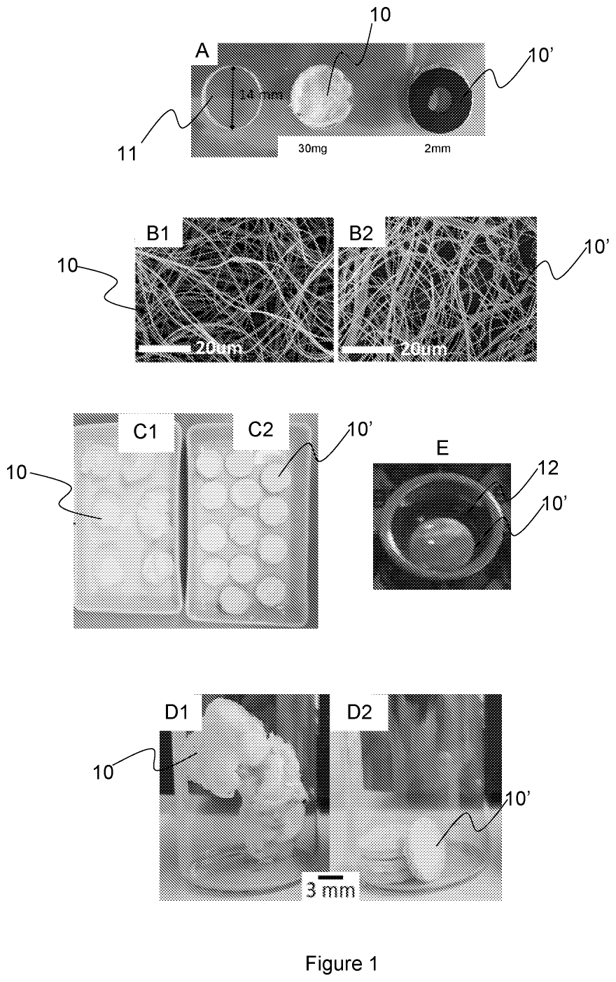

FIG. 1 shows the steps for formation of an implant including: FIG. 1A compression of electrospun fibres, FIG. 1B SEM of as-spun (B1) and compressed fibres (B2); FIG. 1C as-spun fibres after calcining (C1) and compressed fibres after calcining (C2); FIG. 1D as-spun fibres after sterilisation (D1) and compressed fibres after sterilisation (D2); FIG. 1E shows compressed fibres within a cell culture medium;

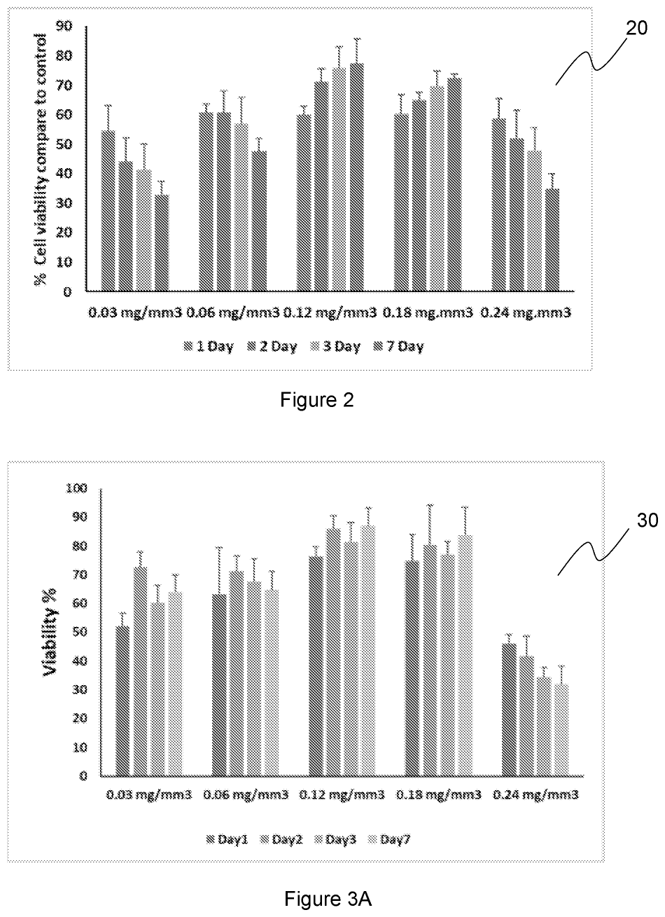

FIG. 2 is a graph of the results of an alamar blue assay on various densities of implant;

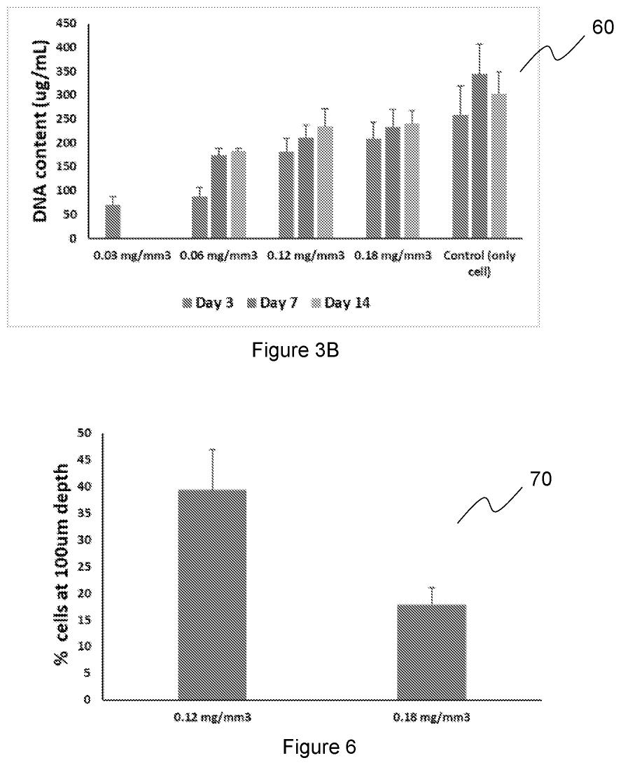

FIG. 3A is a graph of the results of a live/dead assay on various densities of implant;

FIG. 3B is a graph of DNA content over time of various densities of implant compared to control which is cell culture plastic

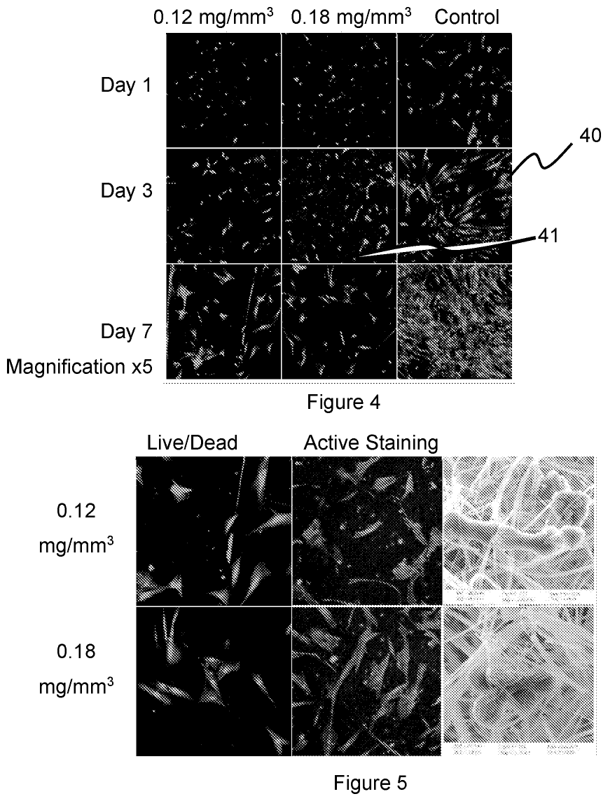

FIG. 4 shows the results of a live/dead assay over time;

FIG. 5 shows the cell morphology at seven days on implants

FIGS. 5A and 5B show SEM images of growth;

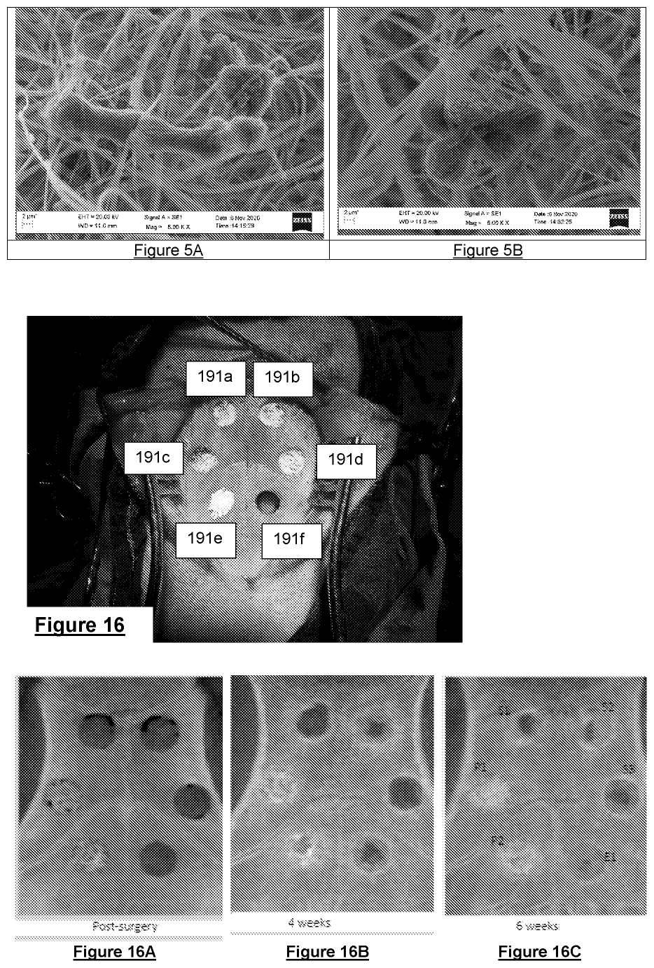

FIG. 6 is a graph of the results of cell migration within various densities of implant;

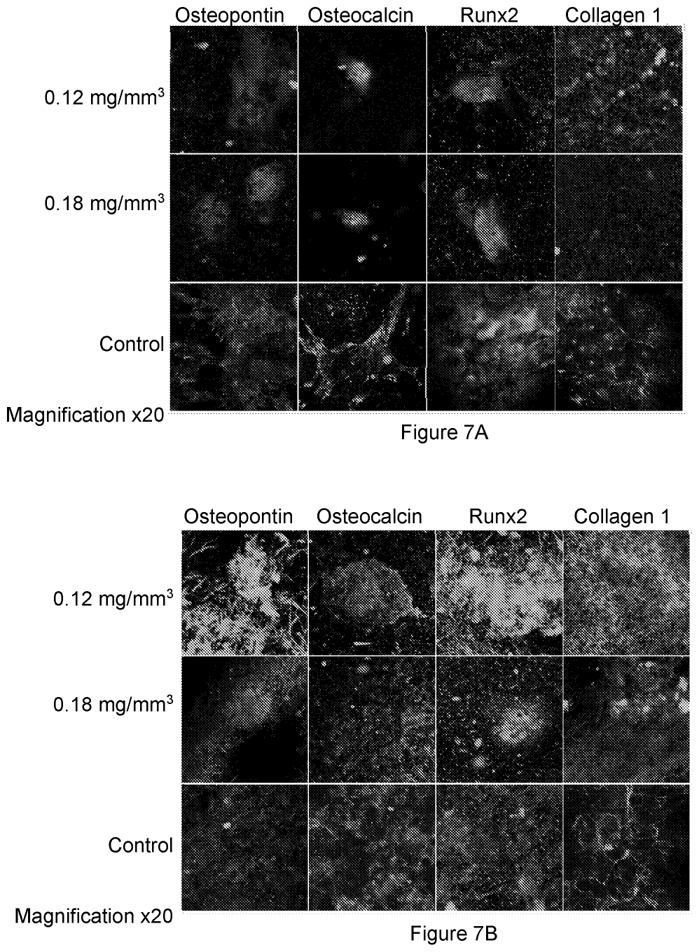

FIGS. 7A and 7B show results for osteogenic markers at 2 and 3 weeks respectively;

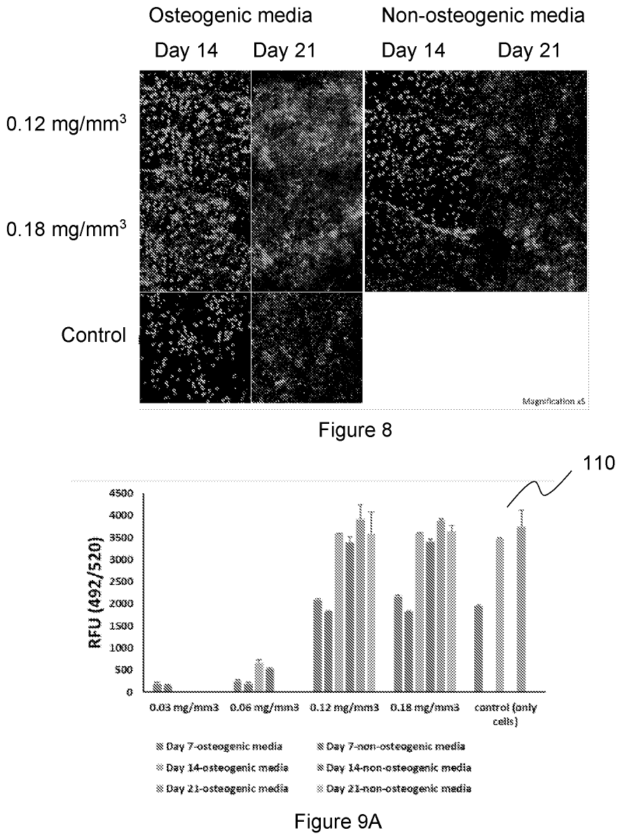

FIG. 8 shows the results from mineralisation studies at day 14 and 21;

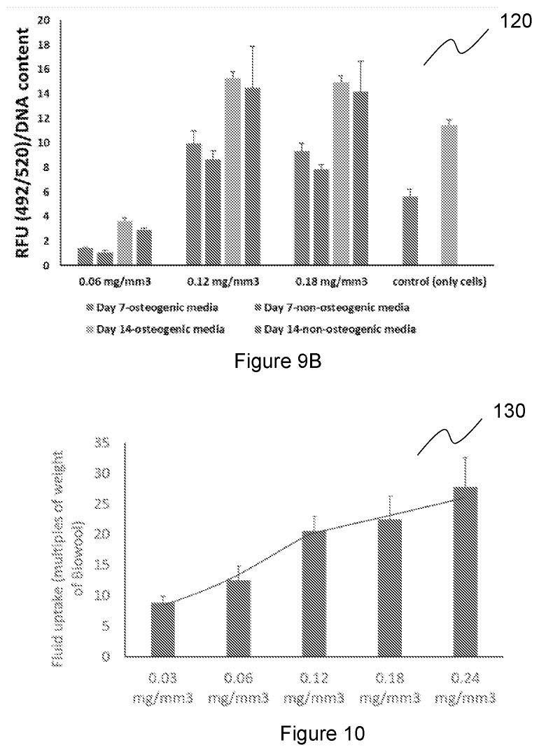

FIGS. 9A and 9B are graphs of the results of mineralization assays;

FIG. 10 is a graph of the results of fluid uptake of implants;

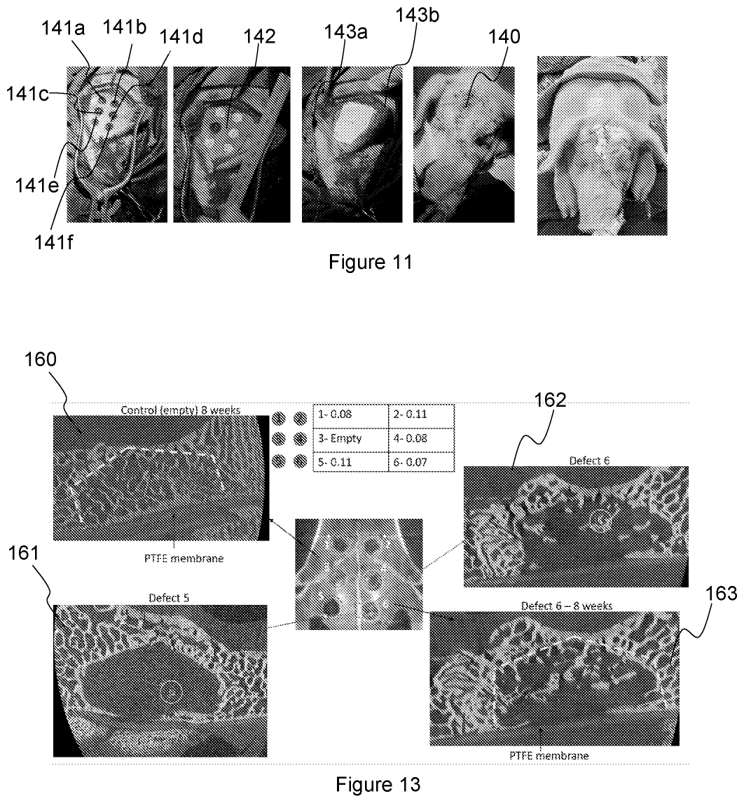

FIG. 11 shows stages of a surgical procedure for Study 1;

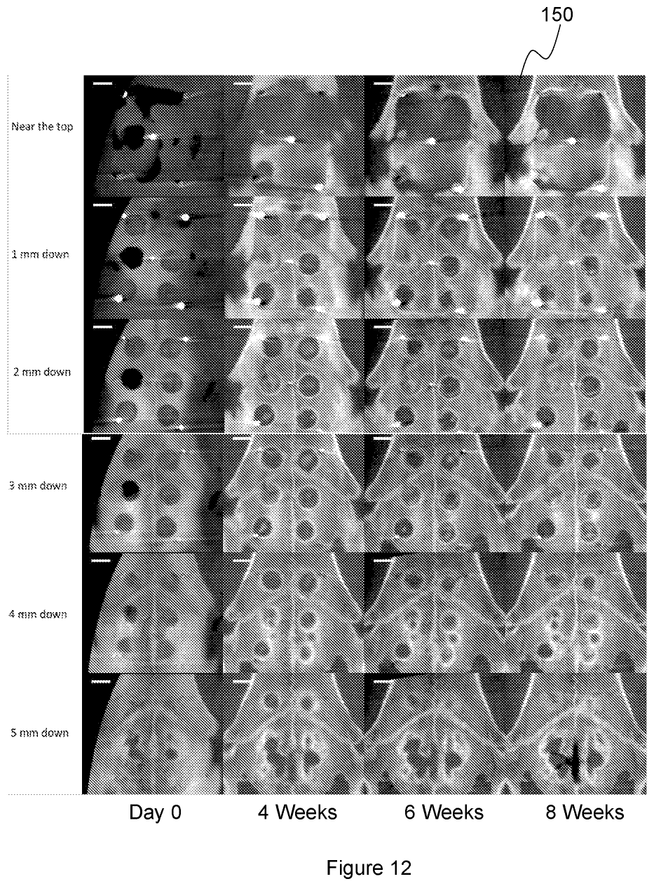

FIG. 12 show CT scans of the progress of implants during Study 1;

FIG. 13 show images of defects at termination;

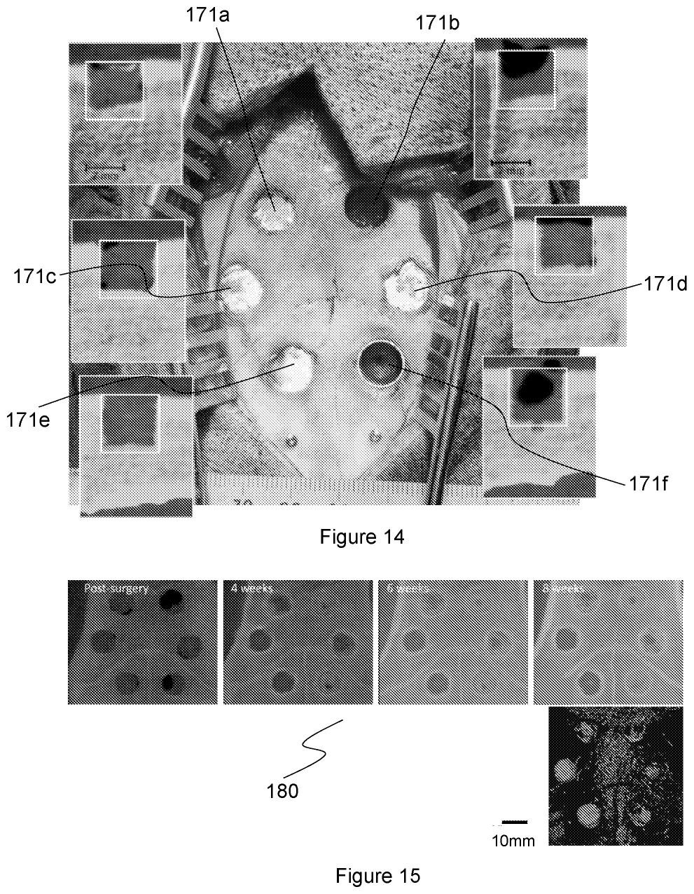

FIG. 14 shows a stage of a surgical procedure for Study 2;

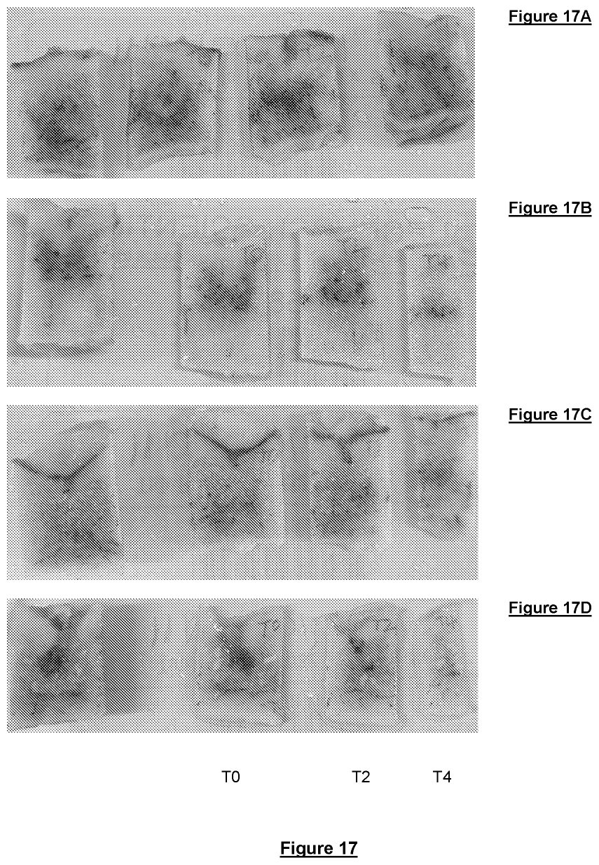

FIG. 15 show CT scans of the progress of implants during Study 2;

FIG. 16 is a photo of location of materials for a comparative study;

FIGS. 16A, B, C are CT scans of the progress of the implants of the comparative study (0, 4, 6 weeks respectively);

FIGS. 17A, B, C, D are photographs of sections through harvested defects;

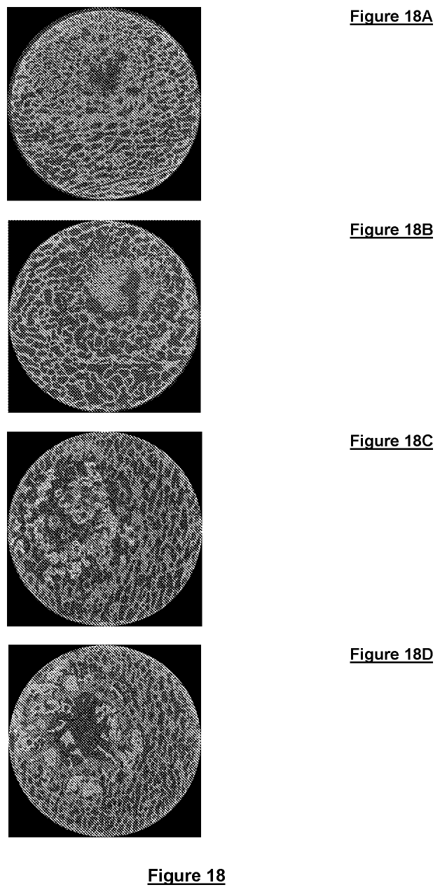

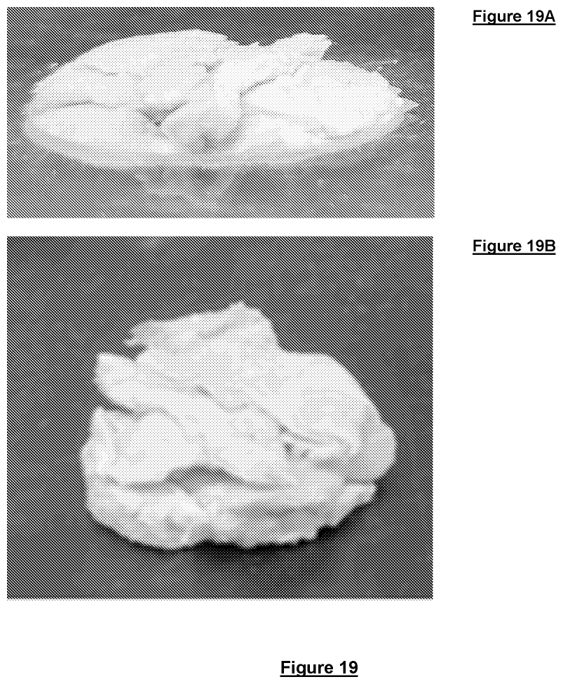

FIGS. 18A, B, C, D are tomography images of horizontal sections through harvested defects; and

FIG. 19A and 19B show comparative examples.

### Preparation of Implants

Implants were prepared according to the following protocol:
a) Sol Gel Preparation
   A sol gel solution was prepared as follows
   In a Teflon beaker add 1.7 ml of 1N HNO₃ to which add 3.7 ml of ethanol and 7.2 ml of tetraethyl orthosilicate (TEOS). After 1 hour add 3.2 g of Ca (NO₃)₂·4H₂O. Leave to mix for 1 h. Then retain at 37° C. for 24 h to form a sol solution.
   In a new beaker add 1 g of polyvinyl butyral (Butvar B-98) to 10 ml of ethanol. Then add 10 ml of this to 10 ml of sol solution, mix well and spin into a box collector to form a sol-gel solution.
b) Electrospinning
   The sol-gel solution was electrospun using the following conditions:
   Spinning conditions of 23 kV, 10 cm distance, flow rate 3.5 ml/h, humidity 38-42% using nozzles with 22 to 32 gauge size.
   The electrospun fibres had a composition of 70 w/w % SiO₂ and 30 w/w % CaO.
   An SEM image of collated, as-spun fibres is shown in FIG. 1B. The fibres have a modal diameter of 800 nm (measured using SEM images and based on 50 samples).
c) Formation
   Referring now to FIG. 1, there is shown the sequential steps for preparing an implant according to the invention.
   As shown in FIG. 1A, 30 mg of the electrospun fibres 10 was placed in a 14 mm diameter die and was compressed to a thickness of 2 mm to form a compressed body or mesh 10′.
   FIG. 1B shows scanning electron microscope (SEM) images of the as-spun fibres 10 (FIG. 1 B1) and the compressed (0.10 g/cm³) fibre mesh 10′ (FIG. 1 B2). It is evident that the fibres of the compressed fibre mesh 10′ are packed much more closely together than the as spun fibres 10 and evidence of inter-fibre bonding is seen at interfibre contacts.
   The compressed fibre mesh 10′ was then heated at a fixed temperature of 65° C. for 3 hours, shown in FIG. 1C2 (FIG. 1C1 shows a comparison with heated as-spun fibres). The resulting fibres were calcinated by sintering at 650° C. for 300 mins at a ramp rate of 1° C./min with a temperature stabilisation step at 300° C. for 30 mins. FIG. 1D2 shows the compressed fibre mesh 10″ after calcination. For comparison a sample of as-spun fibres is shown after calcination (FIG. 1D1). The compressed, heat treated, fibre mash 10″ has retained its shape. After sintering the compressed fibre mesh 10″ reduces to a weight of 11.5 mg, losing roughly 40% of its weight and shrinks to 10 mm in diameter, i.e. the implant has a density of 0.07 g/cm³.
   The compressed fibre mesh 10″ was then sterilised by placing in an oven at 180° C. for 2 h (FIG. 1D2). For comparison a sample of as-spun fibres is shown after sterilisation (FIG. 1D1). No further shape or size changes are found subsequent to sterilisation.
   The fibres of the compressed, heat-treated, fibre mesh 10″ were found to have a porosity of in excess of 90%.

Clearly, different densities can be obtained by using more or less fibres.

### Absorption Capacity of Implants

Referring now to FIG. 1E, there is shown the compressed, heat-treated, fibre mesh 10″ in cell culture 12. The compressed, heat-treated, fibre mesh 10″ was found to support up to 30-40 μL of media which accounts to more than 30-40 times its own weight.
Advantageously, the size of the compressed, heat-treated, fibre mesh 10″ did not change upon absorption of the cell culture, implying that the fibre-fibre bonds generated during the initial heating and subsequent heat-treating stage remain.

Compressed, heat treated, mesh samples 10″ were prepared for in vitro testing and animal studies. In order to identify the optimum fibre packing density and evaluate in vitro osteoblast activity, compressed, heat treated, mesh samples 10″ were formed with different densities.

### In Vitro Testing

In vitro cell-material interactions were tested using assays to monitor cell attachment and migration (confocal and SEM), cytotoxicity (LIVE/DEAD assay), cell proliferation (total DNA), differentiation (ALP and osteocalcin) and bone matrix formation (alizarin-red, Picrosirius-red and FTIR) on pre-calcined compressed mesh samples 10′ with fibre packing densities ranging from 0.03-0.24 g/cm³.

Referring now to FIG. 2, there is shown a graph 20 of an alamar blue assay for compressed mesh samples 10′ with densities of 0.03, 0.06, 0.12, 0.18 and 0.24 mg/mm³. Cell viability was measured and plotted as a percentage of a cell only control sample. Cell viability on 0.12 and 0.18 mg/mm³ samples were shown to be the greatest. Indeed, for the samples at densities of 0.12 and 0.18 mg/mm³ percentage cell viability is shown to increase over time as compared with control, in contrast to the other tested densities.

Referring now to FIG. 3A, there is shown a graph 30 of a live/dead assay for compressed mesh samples 10′ with densities of 0.03, 0.06, 0.12, 0.18 and 0.24 mg/mm³. The cell viability was shown to be dramatically reduced at 0.24 mg/mm³ in comparison to the other samples. Referring now to FIG. 3B, there is shown a graph 60 showing cell proliferation. DNA content is shown to increase with culture, suggesting an increase in cell number that is similar to that observed in tissue culture plastics by Day 3.

### Experimental

Cell proliferation was quantified using PicoGreen dsDNA Assay at 3, 7 and 14 days. Fibre mesh samples 10′ were rinsed twice using DPBS and then they were resuspended in 1 mL of cold lysis buffer for 15 min. To ensure complete cell lysis, samples were vortexed vigorously and subjected to three freeze-thaw cycles. Thereafter, samples were resuspended, and 100 μL of fibre/cell suspension was plated into dark 96 wells, where 100 μL of a working solution of Quant-iT PicoGreen reagent was added. The samples were incubated for 2-5 min at RT. Fluorescence readings were obtained in a plate reader. A standard curve was determined using DNA in serial dilutions. A blank fibre was used to correct the background absorbance, and the assay was performed in triplicate.

At a density of 0.03 mg/mm³ constructs were shown to mostly broken down by 7 days. At a density of 0.06 mg/mm³ constructs were shown to mostly broken down by 14 days. At a density of 0.012 mg/mm³ constructs stayed intact for at least 21 days. Because of the stability of the compressed fibre mesh samples 10′, and because of the cell viability and proliferation data, densities of 0.12 and 0.18 g·cm³ were selected for further analysis.

Referring now to FIG. 4, there is shown a two-colour fluorescence assay (live/dead assay) employed to determine the cell viability on fibers. Samples were washed in PBS before staining by adding 2 μL of ethidium and 2 μL of Calcein to 1 mL of sterile PBS. 100-150 μL of the combined live/dead assay reagent was added to the surface of the fibers and then were left to stain for 45 minutes in the dark. A Zeiss LSM 700 confocal microscope was used for image acquisition. Cell viability was calculated as (number of green stained cells/number of total cells)×100%.

FIG. 4 shows a large number of live cells 40 in comparison to dead cells 41 in the 0.12 mg/mm³, 0.18 mg/mm³ and control samples (cell only). Cells were shown to be rounded on Day 1, by Day 3 the cells had cellular processes with extended filipodia and by Day 7 cells were very well spread on the fibrous samples. This demonstrates that the compressed fibre mesh samples 10′ are viable candidates for implants.

Referring now to FIG. 5, there is shown the cell morphology at 7 days of the 0.12 mg/mm³ and 0.18 mg/mm³ compressed fibre mesh samples 10′. The cells are shown to have spread with extended filopodia on the fibres, as better seen in FIGS. 5A and 5B.

Referring now to FIG. 6, there is shown a graph 70, showing the migration of cells within the matrix of the compressed fibre mesh samples 10′ having a thickness of 200 μm. Cell migration within the 0.12 mg/mm³ sample is greater than that within the 0.18 mg/mm³ sample. Up to 40% of the cells for the 0.12 mg/mm³ sample are at or below the 100 microns depth on day 7, suggesting the mesh is porous and conducive to cell migration.

Referring now to FIG. 7A and 7B, there is shown osteocalcin differentiation at 2 weeks (FIG. 7A) and 3 weeks (FIG. 7B). Osteogenic markers at 2 weeks and 3 weeks were observed in all samples (0.12 mg/mm³, 0.18 mg/mm³ and the cell only control samples).

### Experimental

Cell differentiation was carried out using immunocytochemistry at 14 and 21 days. At each time point, Fibre were rinsed twice with 1×PBS. Thereafter, samples were fixed in 4% (w/V) paraformaldehyde for 20 min at RT. Subsequently, samples were washed three times with 1×PBS and permeabilized and blocked using 0.1% (v/v) Triton X-100, 10% goat serum and 3% BSA in PBS for 1 h at RT. Then, the samples overnight at 4° C. with Rabbit primary antibodies for osteocalcin, osteopontin, RUNX 2 and collagen I. The next day, samples were washed twice with 1×PBS. After the last wash, the samples were incubated for 2 h at RT in the dark with AlexaFluor 488 Goat anti-rabbit secondary antibody. The Samples were finally washed three times with 1×PBS and being mounted using DAPI reagent to stain cell nuclei. Images were obtained using a confocal microscope.

Referring now to FIG. 8, there is shown mineralisation of 0.12 mg/mm³, 0.18 mg/mm³ and cell only control samples in osteogenic and non-osteogenic (basal) media.

### Experimental

The extent of mineralization was determined using an Osteoimage Mineralization Assay at 7,14 and 21 days. After each culture time point, the fibres were washed with 1×PBS before being fixed with 4% (w/v) PFA for 20 min at RT. Samples were subsequently washed further twice (5-10 min each) with Osteoimage wash buffer and then incubated with staining reagent at RT in the dark for 30 min. After incubation, gels were washed three times (5 min each) with wash buffer. To quantify the extent of mineralization, washed samples were resuspended in 500 μL wash buffer and their fluorescence determined in a fluorescent plate reader at a 492/520 nm ratio. Images were obtained using confocal microscope. For comparative, osteogenic media and non-osteogenic (basal) culture media were used for cells. A blank fibre was used to correct the background absorbance.
Osteogenic media contains additional supplements such as B-glycero-phopshate, ascorbic acid and dexamethasone.

Mineralisation was observed in all compressed fibre mesh samples 10′ (0.12 mg/mm^{3, 0.18} mg/mm³ and control samples) from day 14 or earlier. Importantly, compressed fibre mesh samples 10′ is seen to induce bone formation in the basal medium.

Referring now to FIGS. 9A and 9B, there is shown graphs 110, 120 showing the mineralisation of 0.03 mg/mm^{3, 0.06} mg/mm^{3, 0.12} mg/mm^{3, 0.18} mg/mm³ and/or cell only control samples in osteogenic and non-osteogenic media.

### Experimental

Details are the same as discussed above in relation to FIG. 8. However here the fluorescence intensity was quantified to using a fluorescent plate reader at a 492/520 nm ratio. FIG. 9B shows fluorescence intensity normalised to DNA content (FIG. 3B).

The osteolmage™ mineralisation assay (FIG. 9A) shows compressed fibre mesh samples 10′ have a higher potential for matrix deposition than the controls. As demonstrated before, the compressed fibre mesh samples 10′ are seen to induce matrix deposition in the basal and osteogenic medium. The quantity of minerals produced was found to be similar in both medium.

Referring now to FIG. 10, there is shown a graph 130 of fluid uptake by compressed fibre mesh samples 10′. Fluid uptake by biowool increases with increasing packing density.

The above results demonstrate that the compressed, heat treated, mesh samples 10″ are capable of allowing cell proliferation and migration and are suitable candidates for implants for use in the surgical treatment of bone defects.

### In Vivo Studies

In order to further demonstrate that the compressed, heat treated, mesh samples 10″ of the invention are suitable candidates for implants, and specifically implants for the treatment of bone defects a series of in vivo studies were performed on pig heads. Pigs were selected because they have a similar bone mineral density, morphology and healing to that of humans. Further, as pigs have a thicker skull than humans the introduced defects do not penetrate the brain.

### a) Study 1

Referring now to FIG. 11, six identical calvarial defects 141a to 141f were created using trephine in the frontal bone 142 of a pig head 140. An incision was made across the coronal-sagittal plane (“T” shaped), to create 2 flaps 143a, 143b to allow the soft tissue and periosteum to fold back, exposing the frontal bone. The six bone defects 141a to 141f were created in stages, avoiding cranial sutures and the dura, using drill bits of increasing diameter (2-10 mm), and a surgical guide. Saline irrigation was used to prevent raised temperature of the bone, whilst drilling. Two titanium microscrews were placed using a surgical guide, to assist with CT scan alignments.

Each defect 141 is 10 mm in diameter and has a depth of 10 mm Each defect 141 is positioned at least 10 mm apart from each other, to avoid biological interactions between the defects 141.

The defects were filled with compressed, heat treated, mesh samples 10″ as follows: defect 141a 0.08 mg/mm³, defect 141b 0.11 mg/mm³, defect 141c empty, defect 141d 0.08 mg/mm³, defect 141e 0.11 mg/mm³ and defect 141f 0.07 mg/mm³.

The periosteum and soft tissue were then repositioned. The periosteum was not resutured. The skin was sutured in two layers using Vicryl (subcutaneous layer) and Prolene, and veterinary wound powder and op site spray was applied.

FIG. 12 shows CT scans 150 of the defects 141 (shown in FIG. 11) at different depths (near the top i.e. less than 1 mm, 1 mm down, 2 mm down, 3 mm down, 4 mm down and 5 mm down) performed on the day of surgery (Day 0), 4 weeks, 6 weeks and at termination (8 weeks), to quantify bone mineral density and volume at defect. The images demonstrate the progressive formation of bone within the defect over time. It will be appreciated that the empty defect 141c showed relatively rapid healing. It is believed that this is because the defect is not critical due to the young age (<6 months) of the animal at surgery. Axiomatically, in an critical defect the empty cell cannot and will not heal to more than 70% of the original volume within a 52 week time period. We believe that a critical defect in which an implant of the invention is inserted will heal.

Referring now to FIG. 13, there is shown images of defects 141c (empty) image 160, defect 141e (0.11 mg/mm³) image 161 and defect 141f (0.07 mg/mm³) images 162 and 163 after 8 weeks. It will be appreciated that significant ingrowth and bone development has occurred at 8 weeks after installation.

### b) Study 2

In order to study the effect of different compositions of compressed, heat treated, mesh samples 10″ a further study was arranged.

In this study the fibres were either 70:30 SiO₂:CaO (denoted 7030) or 80:20 SiO₂:CaO (denoted 8020). In both cases fibres were electrospun from an appropriate sol-gel composition and compressed and heat treated in accordance with the above-described protocol

Referring now to FIG. 14, six identical calvarial defects 171a to 171f, were created using trephine in the frontal bone 172 of a pig cadaver head 170 as described above in Study 1.

The defects 171 were filled with samples as follows: defect 171a 7030-0.09 mg/mm³, defect 171b empty, defect 171c 8020-0.09 mg/mm³, defect 171d 7030-0.07 mg/mm³, defect 171e 8020-0.07 mg/mm³ and defect 171f empty.

FIG. 15 shows CT scans 180 of the defects 171 (shown in FIG. 17) performed post-surgery (Day 0), 4 weeks, 6 weeks and at termination (8 weeks), to quantify bone mineral density and volume at defect.

The data appears to show greater bone formation within defects 171a and 171d (which were filled with 7030 samples) than within defects 171c and 171e (which were filled with 8020 samples) indicating that higher CaO content may be preferential.

The above data demonstrate that the compressed, heat treated, mesh samples 10″ are able to maintain 3D environment for bone growth. It is believed that the compressed fibre samples 10′ of the invention are likely to be beneficial over particulate materials in challenging (and yet very common) surgical situations, for example where the alveolar ridge of the mandible/maxilla requires lateral and vertical augmentation. In such cases a self-supporting material such as the compressed, heat treated, mesh samples 10″ of the invention will have considerable advantages.

It is also believed that the compressed, heat treated, mesh samples 10″ of the invention are clinically (and/or surgically) beneficial over loose packed fibres because such loose packed fibres, when placed in a bone defect, expand by wicking up blood. This wicking changes the fibre packing density according to the amount of blood present. By compressing and heat treating the fibres it is possible to provide a compressed, heat treated, mesh samples 10″ which has, and which maintains, an optimum fibre packing density for efficient and reproducible bone formation. In addition, an expanding loose packed fibre bundle will fail to provide a stable environment for bone regeneration. Therefore, the above data demonstrate that providing a compressed and bonded fibre structure we can effectively allow, encourage or cause bone ingrowth.

### Comparative Study 1

In order to test the hypothesis a further experiment was conducted in which commercially available implant materials (Bio-Oss and Maxresorb) were compared with compressed, heat treated, mesh samples 10″ of the invention (density 0.07 g/cm³) inserted into cranial defects in a pig.

FIG. 14 shows the location of the defects and of the materials used. In particular:

| Defect | Material |
|---|---|
| 191a | 10′ |
| 191b | 10′ |
| 191c | Maxresorb |
| 191d | 10′ |
| 191e | Bio-Oss |
| 191f | Empty |

BioOss and Maxresorb were used as received from the manufacturer and were located within the respective cavity to an estimated density of 1.63 g/cm³ and 1.60 g/cm³ respectively.

FIG. 16 shows the results of the comparative study. As can be seen, the commercially available materials demonstrate a significant amount of granular material remaining at 6 weeks, whereas the compressed, heat treated, mesh samples 10″ show bone ingrowth, clearly exhibiting a beneficial outcome.

### Comparative Study 2

In order to further determine the performance of materials of the invention compared to clinically-approved materials BioOss and Maxresorb a further set of experiments were performed on 4 pigs.

Cranial defects were provided in each pig as previously described and the defects were filled with compressed, heat treated, mesh samples 10″ of the invention (7030 at 0.7 mg/mm³), BioOss or Maxresorb (as received).

The defects and the surrounding bone (as a 15 mm core) were harvested at 8 weeks to determine the extent of ingrowth of bone.

Reference is made to FIGS. 17 and 18 where FIG. 17 shows vertical sections through the cores in the region of the created defects of animal BW-5, as follows:

| FIG. | Material |
|---|---|
| 17A, 18A | 10′ |
| 17B, 18B | Empty |
| 17C, 18C | Max Resorb |
| 17D, 18D | BioOss |

Where in each case of FIG. 17, TO represents a vertical section along the diameter of the core, T2 represents a vertical section 2 mm from the diameter and T4 represents a vertical section 4 mm from the diameter.

We believe that the sample of the invention (FIG. 17A), shows greater vascularization than any other same, including the empty defect (FIG. 17B). Further, the material of the invention appears to have resorbed completely, as compared to Max Resorb (FIG. 17C) and BioOss (FIG. 17D) which remain as evidenced by white granules (FIG. 17C) and yellow particles (FIG. 17D).

This is also demonstrated in FIG. 18, which is a horizontal section taken through a core at about 4-6 mm depth (i.e. at the mid-point of the core), which shows the clear presence of the Max Resorb (FIG. 18C) and BioOss (FIG. 18D) materials. Further, large defects are also shown in the Max Resorb (FIG. 18C)_and BioOss (FIG. 18D) materials.

It is also of note that the bone growth for the materials of the invention (FIG. 18A) shows much more pronounced radial bone growth towards the centre of the defect, than the two materials of the prior art.

As noted above, because the cranial defects formed in the animals are not critical the empty defect will heal. However, the greater vascularization in the defect filled with the material of the invention and the greater radial growth towards the centre of the defect (FIGS. 17A, 18B) demonstrates a beneficial effect over the empty defect. Indeed, higher amounts of blood vessels indicates and increased bone functionality and hence healthier, and stronger bone ingrowth.

### Comparative Example

In order to assess the performance and applicability of our methodology to the formation of an implant, a comparative Example was conducted.
A solution identical to that described above was electrospun to generate fibres (see “Preparation of implants” steps a) and b)). The fibres were heat treated at 650° C. using an appropriate ramp rate.
The sintered fibres were packed into a mould using compression to an equivalent density as provided above (see FIG. 19A). Upon removal of the mould the fibres did not retain the shape of the mould (FIG. 19B shows the mould with the top compression removed).

Once completely removed from the mould, the shaped fibre body was unable to retain its shape, was friable and was considered unusable as an implant because it was not self-supporting.

Accordingly, we have concluded that the formation of a ‘fibre-only’, or ‘matrix-free’, implant (i.e. that being one in which fibres are not retained or supported in a consolidating matrix, such as a polymer matrix) which is self-supporting is not possible without deploying an initial, relatively low temperature, heating stage.

The first and the most commercially successful bioactive glass, Bioglass® (45S5), has the composition (in mol %) 46.1% SiO₂,24.4% Nₐ2), 26.9% CaO and 2.6% P₂)₅, is osteoconductive and osteostimulative due to the release of silica species and Ca²⁺ions. In a preferred example of our invention we use a simple binary glass composition of silica and calcium oxide (for example 70% SiO₂ 30% CaO (70S30C)) which, when in contact with body fluid, degrades over time releasing silica species and Ca²⁺ions. Degradation products from our material stimulates mesenchymal stem cells (MSCs) to differentiate and produce large amounts of bone matrix within a short time. We also believe that our compressed fibre mesh samples 10′ are capable of being seeded with MSCs ex situ (as well as other species as explained herein) to further promote bone growth and/or healing. We also understand that many different silicon/calcium chemistries can be used, with our without oxides of other materials (phosphorous, sodium etc) and such appropriate electrospinning solutions may be used in the invention to form a desired implant fibre chemistry.

The compressed, heat treated, mesh samples 10″ are conformable for insertion into defects and can be readily shaped without losing structural integrity. Further, we understand that the fibre mesh samples 10′ utilising the binary glass composition show a several fold increase in the quantity and rate of bone formation using stem cells when cultured with media collected from the binary glass composition in comparison to Bioglass®.

Accordingly, the compressed, heat treated, mesh samples 10″ of the invention are promising candidates for the surgical treatment of bone defects of the jaw, which is still a major unmet clinical need. Those defects can be large, complex in shape, and the graft remains unsupported whilst exposed to forces from the oral environment. Advantageously, the self-supporting compressed, heat treated, mesh samples 10″ of the invention may be able to fulfil the need.

Indeed, our data demonstrate that the fibres of our compressed, heat treated, mesh samples 10″ (implants) are nanoporous and are able to form bone-mineral-like hydroxy-carbonate-apatite upon contact with physiological fluids. Ions released from the fibres promote bone tissue formation.

A non-exhaustive list of the possible uses for the compressed, heat treated, mesh samples 10″ (implants) of the invention is as follows:
a flexible 3-D fibrous structure that is ideal for filling complex wound sites.
A bioactive material that mimics ECM to encourage tissue growth and regeneration.
A scaffold material for deep wounds/wounds in difficult to treat places due to natural flex of body (e.g. knees).
A scaffold material for bone regeneration—may be very useful as a material in joint transplants to encourage new bone growth around the implanted material.
A scaffold material for the filling of large holes in teeth.
A scaffold material for strengthening of dental implants (strengthen the jaw bone that the material is implanted into).
Bone/cartilage regeneration in ‘botched’ plastic surgery (for example to use this technology as a scaffold to encourage new tissue formation, for example in nose jobs that have removed too much tissue).
A scaffold for the regeneration of cartilage in joints (e.g. arthritis patients).

It has been demonstrate above that the compressed, heat treated, mesh samples 10″ of the invention have a very large absorption capacity. This can be utilised to provide an implant which has been impregnated with medicaments (e.g. small molecule, biologics, antibiotics, chemotherapeutic agents, metals and salts) or other beneficial agents (proteins, hormones, enzymes, co-factors blood and components thereof, bone marrow, bone marrow aspirate and the like). The self-supporting and wicking structure opens the possibility of locating an implant impregnated with a beneficial agent at a defect, thereby to promote healing and allow bone formation.

It will also be appreciated by those skilled in the art that any number of combinations of the aforementioned features and/or those shown in the appended drawings provide clear advantages over the prior art and are therefore within the scope of the invention described herein.

1. A method of forming an implant for the repair of defects in bone, the method comprising the following Steps:
(a) electrospinning a solution to form electrospun bioactive glass fibres;
(b) compressing the electrospun bioactive glass fibres to form a compressed body;
(c) heating the compressed body to bond the fibres to form a shaped body; and
(d) heat treating the shaped body to form a heat-treated shaped body.

2. A method according to claim 1, wherein Step (b) comprises compressing the electrospun bioactive glass fibres to a density of 0.02 to 0.3 mg/mm³.

3. A method according to claim 1, wherein Step (c) comprises heating to a temperature of less than 100° C.

4-5. (canceled)

6. A method according to claim 1, wherein Step (c) comprises heating the electrospun bioactive glass fibres for in excess of 30 minutes.

7. A method according to claim 1, wherein Step (d) comprises heat treating the shaped body at a temperature of above 250° C.

8-11. (canceled)

12. A method according to claim 1, further comprising sterilising the heat-treated shaped body to form a sterilised heat-treated shaped body.

13. A method according to claim 1, comprising contacting and/or impregnating the heat-treated shaped body with cells or one or more chemical agents,

14. A method according to claim 1, wherein Step (a) comprises electrospinning a solution comprising silicon and calcium wherein the silicon is present in 70 to 80 wt % and the calcium is present in 20 to 30 wt %.

15. A method according to claim 14, wherein Step (a) comprises electrospinning a solution comprising one or more of potassium, phosphorous, magnesium, copper, silver, zinc and/or cobalt.

16. An implant for the repair of defects in bone, the implant comprising a compressed mass of cross linked or bonded, non-woven and non-layered nanofibers, the nanofibers comprising heat-treated bioactive glass fibres.

17. An implant according to claim 16, having a density of 0.02 to 0.3 mg/mm³.

18. A matrix-free implant for the repair of defects in bone, the implant comprising non-woven bonded bioactive glass fibres and having a density of 0.05 to 0.29 mg/mm³.

19. (canceled)

20. A matrix-free implant according to claim 18, wherein the non-woven bonded bioactive glass fibres have a mean diameter of from 500 to 1500 nm.

21. A matrix-free implant according to claim 18, wherein the bioactive glass fibres are calcined.

22. A matrix-free implant according to claim 18, wherein the bioactive glass fibres are porous.

23. A matrix-free implant according to claim 18, having a thickness of from 0.1 to 5 mm.

24. A matrix-free implant according to claim 18, wherein the bioactive glass fibres comprise SiO₂ and CaO and the bioactive glass fibres may comprise a greater weight percentage of SiO₂ than CaO, and are optionally doped with one or more of potassium, phosphorous, magnesium, copper, silver, zinc and/or cobalt.

25-27. (canceled)

28. A method according to claim 2, wherein Step (c) comprises heating to a temperature of less than 100° C.

29. An implant according to claim 16, wherein the bioactive glass fibres are calcined and porous.

30. An implant according to claim 16, having a thickness of from 0.1 to 5 mm.

31. An implant according to claim 16, further comprising cells or one or more chemical agents.
